(19) United States
(12) Patent Application Publication
(10) Pub. No.: US 2004/0214791 A1
(43) Pub. Date: Oct. 28, 2004

(54) High lactose infant nutrition formula

(76) Inventors: Nancy Auestad, Powell, OH (US); Amy C. Boileau, Springfield, OH (US); Joan R. Jacobs, Worthington, OH (US); Russell J. Merritt, Columbus, OH (US)

Correspondence Address: ROSS PRODUCTS DIVISION OF ABBOTT LABORATORIES, DEPARTMENT 108140-DS/1, 625 CLEVELAND AVENUE, COLUMBUS, OH, 43215-1724, US

(21) Appl. No.: 10/423,323

(22) Filed: Apr. 25, 2003

(51) Int. Cl. A61K 31/7012; A61K 47/00

(52) U.S. Cl. 514/53; 424/439

(57) ABSTRACT

Disclosed are infant nutrition formulas comprising protein, lipid and a lactose-containing carbohydrate, wherein the lactose represents from about 68% to about 97% of the total carbohydrate calories in the formula, and wherein the formula preferably provides a caloric density of from about 22 kcal/ounce to about 23 kcal/ounce. Also disclosed are methods of providing nutrition and/or reducing feeding intolerance in preterm infants after hospital discharge by feeding the disclosed compositions to the infants following hospital discharge.

### TECHNICAL FIELD

The present invention relates to high lactose infant nutrition formulas and methods of improving feeding tolerance in premature infants after hospital discharge.

### BACKGROUND OF THE INVENTION

Nothing is more important than providing for the well-being of our children, including meeting their many needs and rising beyond the challenges faced by our infants during their first precious year of life. Among these many challenges, however, few are so great as those faced by the premature infant.

One such challenge faced by our premature infants is feeding intolerance, which can range from mildly uncomfortable to life threatening. This area alone has been a major focus of academic, medical and corporate research over the years, and remains a major research effort, much of which is directed to the development of formulas and methods to improve feeding tolerance among these special children.

Among the many feeding issues associated with premature infants is the high rate of feeding intolerance or difficulties that these infants face following hospital discharge. During the first several weeks after discharge, these infants are often fussy and can easily develop constipation or other mild feeding-related gastrointestinal difficulties that are unpleasant for the infants.

In an attempt to formulate premature infant formulas that would improve feeding tolerance, some researchers have suggested that lactose might be contributing to many tolerance-related feeding problems, and that lactose-free formulas might be useful in this particular application. It has been suggested that premature infants are unable to digest lactose due to a deficiency of lactase activity. Lactase hydrolyzes lactose to glucose and galactose and is detectable at 12 weeks gestation but accumulates slowly so that at 34 weeks gestation, lactase activity is believed to be only 30% that of term infants. In accordance with this view of lactose-related feeding intolerance in premature infants, commercially available formulas designed specifically for premature infants do not generally contain more than 50% of the carbohydrate calories as lactose.

The infant formula literature includes publications and referenced studies that advocate the use of low-lactose formulas in premature infants to improve feeding tolerance. Many of these studies have been directed to premature infant feedings prior to hospital discharge, including early initial in-hospital feedings (see M. Griffin, J. Hansen, Can the elimination of lactose from formula improve feeding tolerance in premature infants, J. Pediatr 1999; 135:587-92.

Unlike most premature infant formulas, human milk derives all of its carbohydrate calories from lactose as do most milk-based term infant formulas. It would therefore be desirable to provide a premature infant formula that would better assimilate the lactose content of human milk, and to provide a method of using such a formula in premature infants to provide optimal nutrition while also minimizing or eliminating feeding intolerance.

It has now been found, however, that many premature infants immediately after an appropriate hospital discharge can not only tolerate a greater proportion of carbohydrate calories as lactose, but they actually exhibit improved tolerance when fed such a formula during the first several weeks after hospital discharge. It has been discovered that an infant nutrition formula comprising from about 68% to about 97%, preferably from about 70% to about 90%, most preferably from 70% to 80%, of lactose as a percentage of the carbohydrate calories in a formula, improves the overall feeding experience of premature infants after hospital discharge, and in particular reduces infant fussiness and decreases constipation as measured by frequency of hard stools and parent perception that their child appeared constipated.

### SUMMARY OF THE INVENTION

The present invention is directed to infant nutrition formulas and methods of using the formulas in premature infants after hospital discharge, wherein the formulas contain from about 68% to about 97% lactose as a percentage of the total carbohydrate calories, and preferably have a caloric density designed for post-discharge premature infants of from about 22 kcal/oz. to 24 kcal/oz.

It has been found that these compositions and methods of the present invention provide premature infants or other similar individuals with improved feeding tolerance following hospital discharge. It has been found that these infants have a sufficient capacity to handle increased lactose loads, to thus allow for the use of the nutrition formulas of the present invention having a lactose concentration closer to that of human milk.

### DETAILED DESCRIPTION OF THE INVENTION

The infant nutrition formulas and corresponding methods of the present invention comprise selectively high lactose concentrations and a preferred caloric density. These and other essential or optional characteristics of the infant nutrition formulas and methods of the present invention are described in detail hereinafter.

The term “infant” as used herein includes children up to one year corrected age, and includes infants from 0 to about 4 months corrected age, infants from about 4 to about 8 months corrected age, infants from about 8 to about 12 months corrected age, low birth weight infants less than 2,500 grams at birth, and premature infants born at less than about 37 weeks gestational age, typically from about 26 weeks to about 34 weeks gestational age.

The term “lipid” as used herein, unless otherwise specified, means fats, oils, and combinations thereof.

The terms “infant formula” and “infant nutrition formula” are used interchangeably herein and refer to nutrition compositions designed for infants, which preferably contain sufficient protein, carbohydrate, lipid, vitamins, minerals, and electrolytes to potentially serve as the sole source of nutrition when provided in sufficient quantities. These formulas, therefore, can be used to provide sole, primary, or supplemental nutrition for the intended infant or infant population.

All percentages, parts and ratios as used herein are by weight of the total composition, unless otherwise specified. All such weights as they pertain to listed ingredients are based on the active level and, therefore, do not include solvents or by-products that may be included in commercially available materials, unless otherwise specified.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, 5, 6, from 1 to 9, from 3.6 to 4.6, from 3.5 to 9.9, and so forth.

All references to singular characteristics or limitations of the present invention shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The nutrition formulas of the present invention can comprise, consist of, or consist essentially of the essential elements and limitations of the invention described herein, as well as any additional or optional ingredients, components, or limitations described herein or otherwise useful in nutrition formula applications.

### Lactose

The infant nutrition formulas of the present invention comprise a lactose-containing carbohydrate component, wherein the lactose represents from about 68% to about 97%, preferably from about 70% to about 90%, even more preferably from about 72% to about 80%, of the carbohydrate calories in the infant formula. Any lactose-containing carbohydrate known for use in infant formulas, or otherwise effective for such use, can be used as a lactose-containing carbohydrate component of the present invention.

Lactose for use in the infant formulas of the present invention are most typically derived or otherwise obtained from cows milk, which generally contains about 5% lactose. The lactose can be added in purified or partially purified form, or added as a fractional ingredient of cows milk or processed cows milk products. Some of the lactose can also be provided as inherent excipients in other added ingredients, e.g., excipient lactose in many protein sources.

The lactose-containing carbohydrate will also contain a minority of non-lactose carbohydrates, non-limiting sources of which include hydrolyzed or intact, naturally and/or chemically modified, starches sourced from corn, tapioca, rice or potato, in waxy or non-waxy forms. Other non-limiting examples of suitable carbohydrate sources include hydrolyzed cornstarch, maltodextrin, glucose polymers, sucrose, corn syrup, corn syrup solids, glucose, fructose, high fructose corn syrup, and combinations thereof.

### Nutrients

The infant nutrition formulas of the present invention comprise sufficient types and amounts of nutrients to help meet the targeted needs of the intended user. These formulas comprise lipid, protein, and carbohydrate, and preferably further comprise one or more of vitamins, minerals, or combinations.

Many different sources and types of carbohydrates, lipids, proteins, minerals and vitamins are known and can be used in the infant formulas of the present invention, provided that such nutrients are compatible with the added ingredients in the selected formulation, are safe and effective for their intended use, and do not otherwise unduly impair product performance. Carbohydrates, including lactose and some non-lactose containing carbohydrates, are described hereinbefore with respect to the lactose requirement of the infant nutrition formula.

The term “protein” as used herein, unless otherwise specified, includes intact and hydrolyzed proteins, free amino acids, and combinations thereof. Proteins suitable for use herein can therefore be hydrolyzed, partially hydrolyzed or non-hydrolyzed, and can be derived from any known or otherwise suitable source such as milk (e.g., casein, whey), animal (e.g., meat, fish), cereal (e.g., rice, corn), vegetable (e.g., soy), or combinations thereof The protein can include, or be entirely or partially replaced by, free amino acids known or otherwise suitable for use in nutritional products, non-limiting examples of which include tryptophan, glutamine, tyrosine, methionine, cysteine, carnitine, arginine, and combinations thereof.

Lipids suitable for use in the nutrition formulas include, but are not limited to, coconut oil, soy oil, corn oil, olive oil, safflower oil, high oleic safflower oil, MCT oil (medium chain triglycerides), sunflower oil, high oleic sunflower oil, structured triglycerides, palm and palm kernel oils, palm olein, canola oil, marine oils, cottonseed oils, and combinations thereof.

Other suitable lipids or related materials include those that provide specific fatty acids, including arachidonic acid, docosahexaenoic acid, and mixtures thereof. These materials are known to provide beneficial effects in infants such as enhanced brain and vision development, descriptions of which are set forth in U.S. Pat. No. 5,492,938 (Kyle et al.), which descriptions are incorporated herein by reference. Non-limiting sources of arachidonic acid and docosahexaenoic acid include marine oil, egg derived oils, fungal oil, algal oil, and combinations thereof.

The infant nutrition formulas may further comprise any of a variety of vitamins, non-limiting examples of which include vitamin A, vitamin D, vitamin E, vitamin K, thiamine, riboflavin, pyridoxine, vitamin B₁₂, niacin, folic acid, pantothenic acid, biotin, vitamin C, choline, inositol, salts and derivatives thereof, and combinations thereof.

The infant nutrition formulas may further comprise any of a variety of minerals known or otherwise suitable for us in infant nutrition formulas, non-limiting examples of which include calcium, phosphorus, magnesium, iron, zinc, manganese, copper, iodine, sodium, potassium, chloride, selenium, and combinations thereof.

The infant nutrition formulas of the present invention preferably comprise nutrients in accordance with the relevant infant formula guidelines for the targeted consumer or user population, an example of which would be the Infant Formula Act, 21 U.S.C. Section 350(a). Preferred carbohydrate, lipid, and protein concentrations for use in the formulas are set forth in the following table.

TABLE 1
Infant Formula Nutrients*

| | | | gm/100 gm | gm/liter |
|---|---|---|---|---|
| Nutrient | Range | gm/100 kcal | powder | as fed |
| Carbohydrate | Preferred | 8-16 | 30-90 | 54-108 |
| | More preferred | 9-13 | 45-60 | 61-88 |
| Lipid | Preferred | 3-8 | 15-35 | 20-54 |
| | More preferred | 4-6.6 | 25—25 | 27-45 |
| Protein | Preferred | 1-3.5 | 8-17 | 7-24 |
| | More preferred | 1.5-3.4 | 10-17 | 10-23 |

*all numerical values are preferably modified by the term “about”

The infant nutrition formula also preferably includes per 100 kcal of formula one or more of the following: vitamin A (from about 250 to about 750 IU), vitamin D (from about 40 to about 100 IU), vitamin K (greater than about 4 μm), vitamin E (at least about 0.3 IU), vitamin C (at least about 8 mg), thiamine (at least about 8 μg), vitamin B₁₂ (at least about 0.15 μg), niacin (at least about 250 μg), folic acid (at least about 4 μg), pantothenic acid (at least about 300 μg), biotin (at least about 1.5 μg), choline (at least about 7 mg), and inositol (at least about 2 mg).

The infant nutrition formula also preferably includes per 100 kcal of formula one or more of the following: calcium (at least about 50 mg), phosphorus (at least about 25 mg), magnesium (at least about 6 mg), iron (at least about 0.15 mg), iodine (at least about 5 μg), zinc (at least about 0.5 mg), copper (at least about 60 μg), manganese (at least about 5 μg), sodium (from about 20 to about 60 mg), potassium (from about 80 to about 200 mg), chloride (from about 55 to about 150 mg) and selenium (at least about 0.5 mcg).

The infant nutrition formula also preferably includes, as part of the lipid component, arachidonic acid and docosahexaenoic acid, which have been shown to have beneficial effects in infants, including enhanced brain and vision development. These lipids and some of their effects are described, for example, in U.S. Pat. No. 5,492,938 (Kyle et al.), which description is incorporated herein by reference. Non-limiting examples of sources of these lipids include marine oil, egg derived oils, fungal oil, algal oil, and combinations thereof.

The infant nutrition formula preferably comprises one or more of arachidonic acid, docosahexaenoic acid, or combinations thereof, alone or in further combination with linoleic acid and linolenic acid. Arachidonic acid concentrations preferably range up to about 2.0%, more preferably from about 0.2% to about 1.0%, even more preferably from about 0.35% to about 0.9%, and most preferably from about 0.4% to about 0.5%, by weight of the total fatty acids in the formula. Docosahexaenoic acid concentrations preferably range up to about 1.0%, more preferably from about 0.1% to about 1.0%, and even more preferably from about 0.19% to about 0.36%, by weight of the total fatty acids in the formula. Linoleic concentrations preferably range up to about 30%, more preferably from about 10% to about 30%, and even more preferably from about 15% to about 20%, by weight of the total fatty acids in the formula. Linolenic acid concentrations preferably range up to about 4%, more preferably from about 1.5% to about 4%, even more preferably from about 2% to about 3%, and even more preferably from about 2.2% to about 2.6%. These preferred lipid materials are described in U.S. Pat. No. 6,495,599 (Auestad et al.), which description is incorporated herein by reference.

### Product Form

The infant nutrition formulas of the present invention can be prepared in any of a variety of product forms, but will most typically be in the form of a ready-to-feed liquid, a liquid concentrate that can be diluted prior to consumption, or a powder that is also to be reconstituted prior to consumption or used to fortify human milk. The finished products are typically packaged and sealed in single or multi-use containers.

The infant nutrition formulas of the present invention can have or otherwise provide a variety of caloric densities depending upon the particular needs of the infant, but will most typically have or otherwise provide a caloric density of at least about 19 kcal/fl oz (660 kcal/liter), more typically from about 20 kcal/fl oz (675-680 kcal/liter) to about 25 kcal/fl oz (820 kcal/liter), even more typically from about 20 kcal/fl oz (675-680 kcal/liter) to about 24 kcal/fl oz (800-810 kcal/liter).

Preferred are the 22-24 kcal/fl oz formulas, most preferably the 22-23 kcal/fl oz formulas, of the present invention, wherein these formulas are especially useful in providing nutrition and minimizing feeding intolerance for preterm and/or low birth weight infants, particularly following hospital discharge.

Less preferred are the 20-21 kcal/fl oz (675-680 to 700 kcal/liter) formulas, which are primarily directed to term infants.

### Method of Use

The present invention is also directed to a method of providing nutrition and reducing feeding intolerance in premature infants after hospital discharge, wherein the method comprises feeding the infants the lactose-containing infant formulas of the present invention, for a period of at least about 1 week, preferably for at least about 1 month, more preferably for at least about 2 months, even more preferably up to about one year, after hospital discharge.

The present invention is also directed to a method of providing nutrition for premature infants after hospital discharge, wherein the method comprises feeding the infants the lactose-containing infant formula of the present invention, preferably for at least 1 week after hospital discharge, more preferably for at least about 1 month after discharge, even more preferably for at least about 2 months after hospital discharge, even more preferably up to about 1 year. In this context, “after hospital discharge” means that the formulas can be started at any time following discharge, preferably within one month, more preferably within one week, of discharge. It is understood, however, that the formulas and methods of the present invention include the initiation of such formulas and methods at any time within the first year after discharge.

In the context of the methods of the present invention, the nutrition provided the infants can be used to meet their sole, primary, or supplemental nutritional needs. For powder embodiments of the present invention, the corresponding method of the present invention further comprises reconstituting the powder with an aqueous vehicle, most typically water or human milk, to form the desired caloric density, which is then orally or enterally fed to the infant to provide the desired nutrition. For powdered infant formula embodiments of the present invention, each is reconstituted with a quantity of water or other suitable fluid such as human milk to produce a volume suitable for one infant feeding, but generally from about 8 grams to about 9 grams of powder are reconstituted with about 55 to about 65 ml of water to produce the desired nutrient densities.

### Optional Ingredients

The infant formulas of the present invention may further comprise other optional components that may modify the physical, chemical, aesthetic or processing characteristics of the formulas or serve as pharmaceutical or additional nutritional components when used in the targeted population. Many such optional ingredients are known for use in food and nutritional products, including infant formulas, and may also be used in the infant formulas of the present invention, provided that such optional materials are compatible with the essential materials described herein, are safe and effective for their intended use, and do not otherwise unduly impair product performance.

Non-limiting examples of such optional ingredients include preservatives, additional anti-oxidants, emulsifying agents, buffers, colorants, flavors, nucleotides and nucleosides, thickening agents, fiber, stabilizers, and so forth.

### Method of Manufacture

The infant formulas of the present invention may be prepared by any known or otherwise effective technique, suitable for making and formulating infant or similar other formulas. Many such methods are described in the relevant arts or are otherwise well known to those skilled in the nutrition formula art.

The infant nutrition formulas of the present invention can be prepared by any of a variety of known or otherwise effective methods. These methods typically involve the initial formation of an aqueous slurry containing carbohydrates, proteins, lipids, stabilizers or other formulation aids, vitamins, minerals, or combinations thereof. The slurry is emulsified, homogenized and cooled. Various other solutions, mixtures or other materials may be added to the resulting emulsion before, during, or after further processing. This emulsion can then be further diluted, sterilized, and packaged to form a ready-to-feed or concentrated liquid, or it can be sterilized and subsequently processed and packaged as a reconstitutable powder (e.g., spray dried, dry mixed, agglomerated).

Other methods for making infant nutrition formulas are described, for example, in U.S. Pat. No. 6,365,218 (Borschel), which description is incorporated herein by reference.

### EXAMPLES

The following examples illustrate specific embodiments of the infant nutrition formula of the present invention. The examples are given solely for the purpose of illustration and are not to be construed as limitations of the present invention, as many variations thereof are possible without departing from the spirit and scope of the invention.

### Example 1

The following example illustrates a powder infant formula of the present invention, which includes the ingredients listed in Table 1.1.

TABLE 1.1
Powder Infant Formula

| Ingredient | Units | Amount per 100 kcal of formula |
|---|---|---|
| Condensed skim milk solids | gm | 4.4-5.5 |
| Whey protein concentrate | gm | 1.46-1.76 |
| Lactose | gm | 3.0-3.6 |
| Corn syrup solids | gm | 2.24-2.68 |
| High oleic safflower oil | gm | 1.3-1.63 |
| Coconut oil | gm | 1.0-1.2 |
| MCT oil | gm | 1.25-1.5 |
| Soy oil | gm | 1.4-1.7 |
| Arachidonic acid oil | mg | 51.3-61.6 |
| Docosahexaenoic acid oil | mg | 19-22.8 |
| Vitamin/mineral/taurine premix | mg | 32.2-38.6 |
| Ascorbic acid | mg | 33-40 |
| Vitamin ADEK premix | mg | 7.5-9.0 |
| L-ascorbyl palmitate | mg | 6.1-7.4 |
| Vitamin A palmitate | mcg | 78-94 |
| Choline chloride | mg | 10.4-12.4 |
| Mixed tocopherols | mg | 2.05-2.45 |
| Beta carotene | mcg | 195-234 |
| L-carnitine | mg | 5.3-6.3 |
| Nucleotide/choline premix | mg | 41.5-49.9 |
| Ferrous sulfate | mg | 6.5-7.9 |
| Magnesium chloride | mg | 25-30 |
| Potassium citrate | mg | 85-102 |
| Sodium chloride | mg | 12.4-14.8 |
| Calcium phosphate | mg | 76.6-92.0 |
| Calcium carbonate | mg | 37.1-44.5 |

The exemplified formula is prepared by initially forming an aqueous slurry by combining the carbohydrates and minerals with a sufficient amount of water while heating (145-155° F.) and agitating the developing slurry, and thereafter maintaining the slurry at 130-145° F. A separate mixture of oils is also prepared by: 1) combining the ingredient oils with agitation and heat (60-175° F.), and thereafter 2) adding the oil soluble vitamins and whey protein to the oil mixture under agitation, and then 3) holding the resulting protein/oil slurry at 120-140° F. The aqueous slurry is combined with the protein/oil slurry with sufficient water under agitation. The condensed skim milk is then diluted with water, and then the aqueous slurry followed by the protein/oil slurry added to the diluted milk. The pH of the resulting mixture is adjusted, if necessary, to 6.45-6.90 using KOH. The resulting mixture is then subjected to high temperature short time (HTST) heat treatment and sufficient (1500-4000 psi) homogenization pressure. The heat-treated mixture is then emulsified through a single stage homogenizer at 900-1100 psi, and thereafter held at 165-185° F. for 16 seconds. The resulting liquid is then homogenized in a two-stage homogenizer at 3900-4100 psi and 400-600 psi, cooled to 34-45° F., and then held at 34 to 45° F. under agitation. Water soluble vitamins, trace minerals, free amino acids, and other similar materials (e.g., nucleotide/choline premix) with sufficient water are added to the cooled liquid, and thereafter the mixture is brought to a final solids concentration adequate for conventional spray drying. The spray-dried product is then packaged and sealed into suitable containers using conventional packaging processes and techniques.

The exemplified product is also formulated in several other product variations, each variation defined by a different lactose level (68%, 75%, 80%, 85%, 90%, or 97% of the carbohydrate calories from lactose). The exemplified formula, and each of the several product variations, are reconstituted with water to form the desired caloric density, typically 20, 21, 22, 23, or 24 kcal/fl oz. For premature infants after hospital discharge, the formula is reconstituted in accordance with appropriate direction by a physician or other suitable health care professional, typically to a density of 22-23 kcal/fl oz, and then used to provide nutrition to the infant for at least one week, preferably at least one month, following hospital discharge. The formula provides improved tolerance during those initial weeks or months following hospital discharge.

### Example 2

The following example illustrates a ready-to-feed infant formula of the present invention, which includes the ingredients listed in the following Table 2.1.

TABLE 2.1
Ready to Feed Infant Formula

| Ingredient | Units | Amount per 100 kcal of formula |
|---|---|---|
| Condensed skim milk solids | gm | 4.4-5.5 |
| Whey protein concentrate | gm | 1.36-1.64 |
| Lactose | gm | 2.89-3.47 |
| Corn syrup solids | gm | 2.29-2.75 |
| Lecithin | mg | 48.7-58.5 |
| Coconut oil | gm | 1.4-1.7 |
| MCT oil | gm | 1.2-1.5 |
| Soy oil | gm | 2.2-2.6 |
| Arachidonic acid | mg | 49.6-60.0 |
| Docosahexaenoic acid oil | mg | 18.4-22.1 |
| Vitamin/mineral/taurine premix | mg | 32.1-38.5 |
| Ascorbic acid | mg | 33-39 |
| Vitamin ADEK premix | mg | 7.5-9.1 |
| Choline chloride | mg | 9.4-11.2 |
| Vitamin A palmitate | mcg | 64.1-76.9 |
| Beta carotene | mcg | 221-266 |
| Ferrous sulfate | mg | 6.7-8.1 |
| Magnesium chloride | mg | 20.6-24.8 |
| Potassium citrate | mg | 121-145 |
| Sodium chloride | mg | 6.6-8.0 |
| Calcium phosphate | mg | 59.6-71.6 |
| Calcium carbonate | mg | 32.7-39.3 |
| L-carnitine | mg | 5.6-6.8 |
| Nucleotide/choline premix | mg | 37.1-44.5 |
| Carrageenan | mg | 10.0-12.1 |

The exemplified formula is prepared by initially forming an aqueous slurry by combining the carbohydrates and minerals with a sufficient amount of water while heating (145-155° F.) and agitating the developing slurry, and thereafter maintaining the slurry at 130-145° F. A separate mixture of ingredient oils is prepared with agitation and heat (60-175° F.). The lecithin, mono- and diglycerides, oil soluble vitamins, carrageenan, and protein powder are then added to the oil mixture with agitation and maintained at 120-140° F. The condensed skim milk component is then diluted with water. The aqueous slurry followed by the protein/oil slurry are added to the diluted milk. The pH of the resulting mixture is adjusted if necessary to 6.45-6.90 using KOH. The resulting mixture is subjected to ultra high temperature (UHT) heat treatment and sufficient (1500-4000 psi) homogenization pressure. The heat-treated liquid is then emulsified through a single stage homogenizer at 900-1100 psi, and thereafter held at 165-185° F. for 16 seconds. The resulting liquid is then homogenized in a two-stage homogenizer at 3900-4100 psi and 400-600 psi, cooled to 34-45° F., and then held at 34-45° F. under agitation. Water soluble vitamins, trace minerals, free amino acids, and other similar materials (e.g., nucleotide/choline premix) with sufficient water are added to the cooled liquid, and thereafter the mixture is brought to the desired solids concentration and a caloric density ranging of from 20-24 kcal/fl oz, preferably from 21-22 kcal/fl oz.

The exemplified product is also formulated in several other product variations, each variation defined by a different lactose level (68%, 75%, 80%, 85%, 90%, or 97% of the carbohydrate calories from lactose). The exemplified formula, and each of the several product variations, are reconstituted with water to form the desired caloric density, typically 20, 21, 22, 23, or 24 kcal/fl oz. The exemplified formula can also be modified during manufacture as a concentrated infant formula suitable for dilution prior to administration to the appropriate caloric density.

### EXPERIMENT

The following experiment was performed in accordance with the lactose-containing formulas and methods of the present invention. As shown from the experimental data set forth hereinafter, these lactose-containing formulas and methods resulted in improved feeding tolerance among premature infants following hospital discharge.

The primary objective of the study was to evaluate tolerance of infant formulas containing different combinations of carbohydrates (high and low lactose) and fats (high and low oleic blends) during the first month after hospital discharge in preterm infants. A two-by-two factorial design was used for this masked, randomized tolerance study. A total of 114 preterm infants with birth weights between 1000 and 2000 g were recruited at or before hospital discharge. Preterm infants began the study on the day of hospital discharge. After one-week of baseline formula feeding with commercially available Similac® NeoSure® Infant Formula, ready-to-feed (RTF), infants were randomized (n=100) to one of four experimental formulas that differed in carbohydrates and fats using a two-by-two study design. The experimental formulas were fed for three weeks.

The four experimental formulas differed in their carbohydrate source (high and low lactose) and fat blend (high and low oleic acid blends). The study formula base was commercially available Similac® NeoSure® Infant Formula (powder) with the following modifications:

1. The high-lactose carbohydrate mixture contained 75% lactose and 25% corn syrup solids.

2. The low-lactose carbohydrate mixture contained 25% lactose and 75% corn syrup solids.

3. The high-oleic oil blend contained 28% soy oil, 27% high oleic safflower oil, 25% MCT oil, and 20% coconut oil.

4. The low-oleic oil blend contained: 30% soy oil, 30% coconut oil, 25% MCT oil, and 15% corn oil.

5. The baseline formula (Similac® NeoSure® RTF) contained 45% soy oil, 30% coconut oil, and 25% MCT oil; and a carbohydrate mixture of 50% lactose and 50% corn syrup solids.

During the 4-week study, parents completed formula intake records, stool records and daily questions pertaining to infant behavior for 3 days during the baseline, for 7 days immediately following randomization, and for 3 days prior to study exit. Parents completed two questionnaires immediately following the baseline period and also at the exit visit: (1) Infant Feeding & Stool Pattern Questionnaire (validated instrument, Jacobs et al 2002) and (2) Formula Satisfaction Questionnaire.

Experimental Results

A total of 114 infants were recruited. Of those recruited, 100 were randomized to one of 4 study products following baseline, and 79 completed the study. Symptoms of intolerance accounted for the majority of early exit reasons for infants who did not complete the study. The completion rate for the high lactose groups was 86% as compared to 72% for the low lactose groups. There were no significant differences in subject demographic characteristics upon entry to the study. Tolerance related exit reasons and attrition rate are shown in the following Table 3.

TABLE 3
Tolerance Related Exit Reasons for Subjects Who Did Not
Complete the Feeding Protocol

| Tolerance Symptom | High Lactose | Low Lactose |
|---|---|---|
| Excessive spit-up, (n)^{†} | 1 | 4 |
| Excessive gas, (n)^{†} | 3 | 6 |
| Constipation, (n)^{†} | 1 | 5 |

^{†}Refers to number of subjects whose parents reported excessive incidence at time of early exit.

The responses to the parent questionnaires shown on the following table (Table 4) were consistent with the lower attrition in the high-lactose groups and the quantitative data indicating the high-lactose formulas were better tolerated by the infants.

TABLE 4
Main Effects for Lactose Level in Experimental Formulas

| Outcome Variable | High-Lactose | Low-Lactose | p-value |
|---|---|---|---|
| Constipation Dimension Score^{†} | 1.87 | 2.28 | 0.0206 |
| Formula Intake, ml/day* | 689 ± 35 | 643 ± 28 | 0.0537 |
| Infant Feeding and Stool Patterns | | | |
| Questionnaire Responses** | | | |
| “My baby fussed or resisted the | 1.6 | 2.2 | 0.0019 |
| bottle while being fed the formula.” | | | |
| “My baby drank the formula within | 1.5 | 2.1 | 0.0031 |
| a reasonable amount of time.” | | | |
| “My baby appeared constipated.” | 1.9 | 2.4 | 0.0384 |
| Formula Satisfaction Questionnaire | | | |
| Reaponses, % of respondents‡ | | | |
| “Would you want to continue using | 6% reported | 19% reported | 0.0109 |
| the formula?” | “definitely not” | “definitely not” | |
| “Did baby seem to like formula?” | 0% reported “baby | 18% reported “baby | 0.0162 |
| | disliked” | disliked” | |

^{†}Constipation Dimension Score was computed from responses to questions on the Infant Feeding and Stool Patterns Questionnaire. Baseline response was included as a covariate in the statistical model. For this score, the lower the value, the more favorable the score.
*Subjects in the high lactose groups consumed more formula, on average, than subjects in the low lactose groups. Values represent mean ± SEM at study exit.
**For the Infant Feeding and Stool Patterns Questionnaire, parents were asked to score each question on a scale from one to five. All responses were scaled so that a score of 1.0 is the most favorable score possible and 5.0 is the least favorable score. Values were computed from responses at study exit.
‡For the Formula Satisfaction Questionnaire, parents were asked questions designed to assess their satisfaction with the formula as well as the perception of their infant's satisfaction during the feedings. All responses are reported as percent of respondents. Values were computed from responses at study exit.

Conclusion

Preterm infants who were randomized to the high lactose formulas experienced improved tolerance as measured by reduced attrition, fewer reports of tolerance related symptoms, less constipation, increased formula intake, and improved parental responses to a number of items on the two referenced questionnaires. The finding that the higher lactose formulas were consistently associated with more favorable outcomes was unexpected given the large amount of research focused on improving tolerance by replacing or reducing lactose concentrations with glucose polymers and related carbohydrates in infant formulas intended for premature infants.

In short, these findings were especially surprising in view of prevailing theories on the use of low-lactose formulas in some infants to improve feeding tolerance. In contrast to such prevailing theories, our findings as set forth herein now show a nearly opposite result, wherein higher lactose concentrations in premature infant formulas can actually improve the overall feeding experience of premature infants after hospital discharge, which includes reduced infant fussiness and decreased constipation as measured by frequency of hard stools and parent perception that their child appeared constipated.

What is claimed is:

1. A infant nutrition formula comprising protein, lipid and a lactose-containing carbohydrate, wherein the lactose represents from about 68% to about 97% of the total carbohydrate calories in the formula, and wherein the formula provides a caloric density of from about 22 kcal/fl oz to about 24 kcal/fl oz.

2. The nutrition formula of claim 1, wherein the lactose represents from about 70% to about 90% of the carbohydrate calories in the formula.

3. The nutrition formula of claim 1, wherein the lactose represents from about 72% to about 80% of the carbohydrate calories in the formula.

4. The nutrition formula of claim 2, wherein the caloric density ranges from about 22 kcal/fl oz to about 23 kcal/fl oz.

5. The nutrition formula of claim 1, wherein the formula comprises from about 3 grams to about 8 grams of lipid per 100 kcal of the formula.

6. The nutrition formula of claim 1, wherein the formula comprises from about 1 to about 3.5 grams of protein per 100 kcal of the formula.

7. The nutrition formula of claim 1, wherein the formula comprises, per 100 kcal of formula:
(A) from about 3 grams to about 8 grams of lipid;
(B) from about 1 gram to about 3.5 grams of protein, and
(C) from about 8 grams to about 16 grams of carbohydrate.

8. The nutrition formula of claim 1, wherein the formula comprises, per 100 kcal of formula:
(A) from about 4 grams to about 6.6 grams of lipid;
(B) from about 1.5 gram to about 3.4 grams of protein, and
(C) from about 9 grams to about 13 grams of carbohydrate.

9. The nutrition formula of claim 1, wherein the formula is a ready-to-feed liquid.

10. The nutrition formula of claim 1, wherein the formula is a nutritional powder.

11. An infant nutrition formula comprising protein, lipid and a lactose-containing carbohydrate, wherein the lactose represents from about 68% to about 97% of the total carbohydrate calories in the formula.

12. The nutrition formula of claim 11, wherein the lactose represents from about 70% to about 90% of the carbohydrate calories in the formula.

13. The nutrition formula of claim 11, wherein the lactose represents from about 72% to about 80% of the carbohydrate calories in the formula.

14. The nutrition formula of claim 11, wherein the caloric density ranges from about 22 kcal/fl oz to about 23 kcal/fl oz.

15. The nutrition formula of claim 11, wherein the formula comprises from about 3 grams to about 8 grams of lipid per 100 kcal of the formula.

16. The nutrition formula of claim 11, wherein the formula comprises from about 1 to about 3.5 grams of protein per 100 kcal of the formula.

17. The nutrition formula of claim 11, wherein the formula comprises, per 100 kcal of formula:
(A) from about 3 grams to about 8 grams of lipid;
(B) from about 1 gram to about 3.5 grams of protein, and
(C) from about 8 grams to about 16 grams of carbohydrate.

18. The nutrition formula of claim 11, wherein the formula comprises, per 100 kcal of formula:
(A) from about 4 grams to about 6.6 grams of lipid;
(B) from about 1.5 gram to about 3.4 grams of protein, and
(C) from about 9 grams to about 13 grams of carbohydrate.

19. A method of providing nutrition and reducing feeding intolerance in premature infants, said method comprising feeding the infants the formula of claim 1 after hospital discharge.

20. A method of providing nutrition and reducing feeding intolerance in premature infants, said method comprising feeding the infants the formula of claim 1 for a period of at least about 1 week following hospital discharge.

21. A method of providing nutrition and reducing feeding intolerance in premature infants, said method comprising feeding the infants the formula of claim 1 for a period of at least about 2 months following hospital discharge.

22. A method of providing nutrition for premature infants, said method comprising feeding the infants the formula of claim 1 after hospital discharge.

23. A method of providing nutrition and reducing feeding intolerance in premature infants, said method comprising feeding the infants the formula of claim 11 after hospital discharge.

24. A method of providing nutrition and reducing feeding intolerance in premature infants, said method comprising feeding the infants the formula of claim 11 for a period of at least about 1 week following hospital discharge.

25. A method of providing nutrition and reducing feeding intolerance in premature infants, said method comprising feeding the infants the formula of claim 11 for a period of at least about 2 months following hospital discharge.

26. A method of providing nutrition for premature infants, said method comprising feeding the infants the formula of claim 11 after hospital discharge.
